(19) United States
(12) Patent Application Publication
(10) Pub. No.: US 2008/0039483 A1
(43) Pub. Date: Feb. 14, 2008

(54) Novel Chelidonine Derivatives, Methods for the Production Thereof, and Use Thereof For Producing Pharmaceutical Agents

(76) Inventor: Zoser B. Salama, Berlin (DE)

Correspondence Address: JOYCE VON NATZMER;PEQUIGNOT + MYERS LLC, 200 Madison Avenue, Suite 1901, New York, NY, 10016, US

(21) Appl. No.: 10/595,699

(22) PCT Filed: Nov. 5, 2003
(86) PCT No.: PCT/DE03/03702

§ 371 Date: Mar. 27, 2007

(51) Int. Cl. A61K 31/4355; A61P 35/00; A61P 35/02; C07D 491/153

(52) U.S. Cl. 514/279; 546/41

(57) ABSTRACT

The invention relates to novel chelidonine derivatives, methods for the production thereof, the use of said compounds for the prevention, treatment, monitoring of the progress, and aftertreatment of diseases related to cell growth, cell differentiation, and/or cell division, especially tumors, and a kit comprising the inventive chelidonine derivatives.

The invention relates to novel chelidonine derivatives and methods for the production thereof; the invention also relates to the use of said compounds in prophylaxis, therapy, follow-up and aftercare of diseases associated with cell growth, cell differentiation and/or cell division, especially tumors, and to a kit comprising said chelidonine derivatives.

It is well-known that substances contained in various officinal plants, such as milk thistle, mistletoe, black cumin, coneflower, rampion, but also celandine (Chelidonium majus), can be used for various types of diseases. Thus, celandine is effective against warts and can be used in various bilious diseases. Celandine contains about 30 alkaloids with an overall content of about 0.1 to 1 wt.-%, including, among other things, chelelerythrine, sanguinhrine, berberine and chelidonine. Chelidonine is known to have a spasmolytic and analgesic effect. Furthermore, it is known that the yellow milky sap of a fresh plant, owing to its content of chelidonine, is capable of inhibiting cell division. However, the effect on division of cells can be observed in many cell cultures, e.g. in tumorous and non-tumorous cells. For this reason, attempts have been made to provide chelidonine derivatives having a specific effect against tumor cells, but only low or no effect at all on non-degenerate, i.e. non-tumorous cells. Thus, Jalilian et al. (2001) describe synthetic chelidonine derivatives possibly usable as antitumor active substances. These derivatives are fluorobenzoates labelled in a way so as to allow investigations on the interaction with microtubular structures. Grynkiewicz et al. (2001) describe various chelidonine derivatives having effects on the central nervous system. The derivatives disclosed exhibit an anti-serotoninergic effect which has not been disclosed for the chelidonine parent substance.

Another chelidonine derivative is okraine which, as a trimeric compound from celandine alkaloids, is used together with thiophosphoric triazide in cancer therapy. As has been demonstrated, antitumor effectiveness of okraine has been detected in cell cultures and in animal experiments. Furthermore, some disclosures reveal that okraine has a therapeutic effect in humans in cases of prostate carcinomas, colorectal carcinomas and breast cancer. To date, however, it has not been possible to determine which active substances included in different lots of okraine are responsible for the above-described antitumor effect. For the time being, none of the agents being used has been officially approved in the EU member states.

Apart from the doubts with respect to the structures of these substances and also regarding the compositions, there is no proof of effectiveness for most of the substances being used (Hopf, 2002).

The object of the invention was therefore to provide further chelidonine derivatives which could be unambiguously determined in their structure and proven to be the cause of anti-tumoral effectiveness.

The invention solves the above problem by providing new chelidonine derivatives having an anti-tumoral effect, selected from the group comprising chelidonine acetate, chelidoninyl trifluoroacetate, chelidoninyl trichloromethyl carbonate, chelidoninyl methyl succinate, chelidoninyl ethyl oxalate, N-(3-trifluoromethylphenyl)chelidoninylurethane, phenylalanine chelidoninyl ester, proline chelidoninyl ester and/or alanine chelidoninyl ester.

Surprisingly, it was possible to demonstrate that the chelidonine derivatives which were synthesized have anti-tumoral effectiveness.

The invention also relates to pharmaceutical agents comprising the chelidonine derivatives according to the invention, optionally together with a pharmaceutically tolerable carrier, an adjuvant and/or vehicle.

The terms “chelidonine derivatives”, “compounds according to the invention” and “pharmaceutical agents” will be used as synonyms, i.e., where statements as to the compounds according to the invention or chelidonine derivatives are made, they also relate to pharmaceutical agents comprising these structures. The chelidonine derivatives or compounds or pharmaceutical agents according to the invention can be contacted with an organism in a therapeutic amount.

The expression “therapeutic amount” as used herein refers to an amount that prevents or improves symptoms of a disorder or of a responsive, pathologically physiological condition. In specific embodiments of the present invention the amount administered is sufficient to inhibit a tumor in its growth, said amount essentially preventing or inhibiting spreading of a tumor, tumor angiogenesis, tumor invasion and/or tumor metastasizing in a recipient. Accordingly, the invention relates to pharmaceutical agents or drugs comprising the compounds of the invention, optionally together with pharmaceutical adjuvants.

The amount of compounds of the invention to be used in a healthy person in the event of prophylaxis or in a patient in the event of therapy is formulated and the dose established according to conventional medical practice, considering the disorder to be treated, the condition of each individual patient, the site of administration, the procedure of administration and other factors well-known to the attending physicians. Similarly, the dose of the administered compounds of the invention depends on the characteristics of the tumor, on the in vivo half-life of the compounds of the invention in plasma, and on the concentration of the compounds of the invention in the formulation, and also on the route of administration, site and rate of dosage, clinical tolerance of each individual (human and animal), pathological affection of the patient and the like, as is well-known to physicians or other persons skilled in the art. In general, dosages of about 0.1 to 1000 mg per individual and administration are preferred; particularly preferred is a dosage of from 10 to 500 mg, even more preferably 200 to 400 mg, and particularly 300 mg. It is also possible to employ varying dosages during a sequence of consecutive administrations.

For example, injections (intramuscular or subcutaneous or into blood vessels) are envisaged as a route of therapeutic administration of the compounds of the invention, e.g. encapsulated or carrier-bound compounds of the invention, although supply in the form of an aerosol, via catheters or surgical tubes is also applicable. Other preferred routes include suspensions, tablets, capsules and the like for oral administration, commercially available nebulizers for liquid formulations and inhalation of lyophilized or aerolyzed compounds and suppositories for rectal or vaginal administration. Liquid formulations can be The suitability of the selected parameters, e.g. dosage, regimen, selection of adjuvants and the like can be determined by taking serum aliquots from the patient, i.e. human or animal, and testing during the course of the applications. Alternatively or concomitantly, the amount of T cells or other cells of the immune system can be determined in a conventional manner so as to obtain an overall survey of the patient's immunologic constitution. In addition, the clinical condition of the patient can be observed for the desired effect. In particular, growth and metastasizing of tumors can be determined. As tumors can be associated with other diseases, e.g. infections, additional co-monitoring of the latter is also possible.

In general, both aqueous formulations and dry compounds of the invention can be mixed with an excipient so as to provide a stabilizing effect prior to treatment e.g. with a solvent. An aqueous solution of a compound according to the invention can be an inventive compound in suspension or a solution.

The compound of the invention can be incorporated in a solution together with a preservative. Examples of suitable preservatives of suspensions or solutions include phenol, benzyl alcohol, m-cresol, methylparaben, propylparaben, benzalkonium chloride and benzethonium chloride. In general, the formulations of the compounds according to the invention may include components in amounts that will not adversely affect the production of stable forms, and in amounts suitable for effective, safe pharmaceutical administration. For example, other pharmaceutically acceptable excipients well-known to those skilled in the art may form part of the compounds or formulations according to the invention. For example, these include salts, various fillers, additional buffer agents, chelating agents, antioxidants, co-solvents and the like.

In a preferred embodiment of the invention the inventive compounds are associated with liposomes, siosomes and/or niosomes.

For example, this can be accomplished in such a way that the compound according to the invention is entrapped in a liposome or anchored on the surface of a liposome. It is well-known to those skilled in the art that artificial or natural membranes of liposomes may have an immune-stimulating effect, especially in those cases where the components are coupled to the surface of liposomes or entrapped inside the liposomes or simply mixed together with the liposomes. Such formulations of liposomes can be applied on the parenteral route. Using well-known methods, e.g. a spray, such formulations can be applied nasally on the mucosa of the nasal cavity. In a preferred fashion, therapeutic treatment using a spray is suitable for treating lung cancer or tumors in the ear-nose-throat region. Especially in nasal administration, the compound of the invention must be applied on the mucosa in a state permitting penetration of the mucosa or absorption thereby. For this reason, the vesicle must be biocompatible with the mucus and have a certain degree of hydrophilicity. For example, such structures are known to those skilled in the art from EP 0 682 528, the teaching of which is hereby incorporated in the disclosure of the invention. The liposomal composition may comprise one or more additional pharmaceutical carriers selected from surface-active substances and absorption-promoting agents such as polyoxyethylene alcohol ethers, bile salts and derivatives thereof, fusidinic acid and derivatives thereof, oleic acid, lecithin, lysolecithins, Tween® 21 to 85, etc., water-absorbing polymers such as glycofurol, polyethylene glycol 200 to 7500, polyvinylpyrrolidone, propylene glycol or polyacrylic acid, gelatin, cellulose and derivatives etc.; substances inhibiting enzymatic degradation, such as aprotinin etc.; organic solvents such as alcohols, e.g. ethanol, glycerol, benzyl alcohol etc.; or ethyl acetate etc.; hydrophobic agents such as vegetable oil, soybean oil, peanut oil, coconut oil, corn oil, olive oil, sunflower oil, “miglyols” or mixtures thereof, etc.; pH regulators such as nitric acid, phosphoric acid, acetic acid, citrates, etc.; preservatives and agents regulating the osmotic pressure, such as glycerol, sodium chloride, methyl paraoxybenzoate, benzoic acid, etc.; liposomes and/or emulsion formulations such as lecithins etc.; micro-encapsulated formulations; propellants such as butane.

It is preferred in another embodiment of the invention that the compounds according to the invention are optionally associated with each other or, coupled to a carrier, enclosed in liposomes, and such enclosure in liposomes does not necessarily imply—in the meaning of the invention—that the compounds of the invention are present inside the liposomes. Enclosure in the meaning of the invention may also imply that the compounds of the invention are associated with the membrane of the liposomes, e.g. in such a way that the compounds are anchored on the exterior membrane. Such a representation of the inventive compounds in or on liposomes is advantageous in those cases where a person skilled in the art selects the liposomes such that the latter have an immune-stimulating effect. Various ways of modifying the immune-stimulating effect of liposomes are known to those skilled in the art from DE 198 51 282. The lipids can be ordinary lipids, such as esters and amides, or complex lipids, e.g. glycolipids such as cerebrosides or gangliosides, sphingolipids or phospholipids.

In the meaning of the invention, the carriers which can be components of drugs comprising the compounds of the invention can be proteins stimulating an antibody response as a result of their immunogenic behavior, but also pharmaceutical adjuvants well-known to those skilled in the art, such as QS-21, GPI-0100 or other saponines, water-oil emulsions such as Montanides, polylysine, polyarginine compounds, or others, e.g. phosphate-buffered saline, water, various kinds of detergents, sterile solutions and the like.

A pharmaceutical agent in the meaning of the invention is any agent in the field of medicine, which can be used in prophylaxis, diagnosis, therapy, follow-up or aftercare of patients comprising a tumor in such a way that a pathogenic modification of their overall condition or of the condition of particular regions of the organism could establish at least temporarily. Thus, for example, the pharmaceutical agent in the meaning of the invention can be a vaccine or a therapeutic agent. In addition to the compounds of the invention, the pharmaceutical agent in the meaning of the invention may include e.g. an acceptable salt or components thereof. For example, these can be salts of inorganic acids such as phosphoric acid or salts of organic acids.

Furthermore, the salts can be free of carboxyl groups and derived from inorganic bases such as sodium, potassium, ammonium, calcium or iron hydroxides, or from organic bases such as isopropylamine, trimethylamine, 2-ethylaminoethanol, histidine and others. Examples of liquid carriers are sterile aqueous solutions including no further materials or active ingredients, e.g. water, or those comprising a buffer such as sodium phosphate with a physiological pH or a physiological salt solution or both, such as phosphate-buffered sodium chloride solution. Other liquid carriers may comprise more than just one buffer salt, e.g. sodium and potassium chlorides, dextrose, propylene glycol, polyethylene glycol, or others. Liquid compositions of the pharmaceutical agents may additionally comprise a liquid phase, with water being excluded, however. Examples of such additional liquid phases are glycerol, vegetable oils, organic esters or water-oil emulsions. The pharmaceutical composition or pharmaceutical agent typically includes a content of at least 0.1 wt.-% of compounds according to the invention, relative to the overall pharmaceutical composition. The respective dose or dosage range for administering the pharmaceutical agent according to the invention is sufficiently high or wide in order to achieve the desired prophylactic or therapeutic effect of forming neutralizing antibodies. In this context, the dose should not be selected in such a way that undesirable side effects would dominate. In general, the dose will vary with the patient's age, constitution, sex and, of course, depending on the severity of the disease. The individual dose can be adjusted both with reference to the primary disease and with reference to the occurrence of additional complications. Using well-known means and methods, the exact dose can be determined by a person skilled in the art, e.g. by determining the tumor growth as a function of dosage or as a function of the application regime or pharmaceutical carrier and the like. Depending on the patient, the dose can be selected individually. For example, a dose of pharmaceutical agent just tolerated by a patient can be such that the range thereof in plasma or locally in particular organs is from 0.1 to 10,000 μM, preferably between 1 and 100 μM. Alternatively, the dose can be calculated relative to the body weight of the patient. In this event, a typical dose of pharmaceutical agent would have to be adjusted e.g. in a range between 0.1 μg and 100 μg per kg body weight, preferably between 1 and 50 μg/kg. Furthermore, however, it is also possible to determine the dose on the basis of particular organs rather than the whole patient. For example, this would be the case when placing the pharmaceutical agent according to the invention, e.g. in a biopolymer incorporated in the respective patient, near specific organs by means of surgery. Several biopolymers capable of liberating peptides or recombinant proteins in a desirable manner are known to those skilled in the art. For example, such a gel may include 1 to 1000 μg of amino acid sequences of the invention, e.g. peptides or recombinant proteins, or of pharmaceutical agent per ml gel composition, preferably between 5 and 500 μg/ml, and more preferably between 10 and 100 mg/ml. In this event, the therapeutic agent is administered as a solid, gel-like or liquid composition.

In another preferred embodiment of the invention, the carriers are selected from the group of fillers, diluents, binders, humectants, disintegrants, dissolution retarders, absorption enhancers, wetting agents, adsorbents and/or lubricants.

The fillers and diluents are preferably starches, lactose, cane-sugar, glucose, mannitol and silica, the binder is preferably carboxymethylcellulose, alginate, gelatin, polyvinylpyrrolidone, the humectant is preferably glycerol, the disintegrant is preferably agar, calcium carbonate and sodium carbonate, the dissolution retarder is preferably paraffin, and the absorption enhancer is preferably a quaternary ammonium compound, the wetting agent is preferably cetyl alcohol and glycerol monostearate, the adsorbent is preferably kaolin and bentonite, and the lubricant is preferably talc, calcium and magnesium stearate, a solid polyethylene glycol or concerns mixtures of the materials mentioned above.

In another preferred embodiment of the invention the inventive compounds are prepared as gel, poudrage, powder, tablet, sustained-release tablet, premix, emulsion, brew-up formulation, drops, concentrate, granulate, syrup, pellet, bolus, capsule, aerosol, spray and/or inhalant and/or inhalant and/or applied in this form. The tablets, coated tablets, capsules, pills and granulates can be provided with conventional coatings and envelopes optionally including opacification agents, and can be composed such that release of the active substance(s) takes place only or preferably in a particular part of the intestinal tract, optionally in a delayed fashion, to which end polymer substances and waxes can be used as embedding materials.

For example, the drugs of the present invention can be used in oral administration in any orally tolerable dosage form, including capsules, tablets and aqueous suspensions and solutions, without being restricted thereto, however. In case of tablets for oral application, carriers frequently used include lactose and corn starch. Lubricants such as magnesium stearate are typically added. For oral administration in the form of capsules, useful diluents include lactose and dried corn starch. In oral administration of aqueous suspensions the active substance is combined with emulsifiers and suspending agents. Also, particular sweeteners and/or flavors and/or coloring agents can be added, if desired.

The active substance(s), i.e., the compounds of the invention, optionally can be present in a micro-encapsulated form, together with one or more of the above-mentioned carrier substances.

In addition to the active substance(s), suppositories may include conventional water-soluble or water-insoluble carrier substances, e.g. polyethylene glycols, fats, e.g. cocoa fat and higher esters (for example, C₁₄ alcohol with C₁₆ fatty acid) or mixtures of such materials.

In addition to the active substance(s), ointments, pastes, creams and gels may include conventional carrier substances, e.g. animal and vegetable fats, waxes, paraffins, starch, tragacanth, cellulose derivatives, polyethylene glycols, silicones, bentonites, silica, talc and zinc oxide or mixtures of these materials.

In addition to the active substance(s), powders and sprays may include conventional carriers such as lactose, talc, silica, aluminum hydroxide, calcium silicate and polyamide powder or mixtures of these substances. In addition, sprays may include conventional propellants such as chlorofluorohydrocarbons.

In addition to the active substance(s), solutions and emulsions may include conventional carriers such as solvents, solubilizers, and emulsifiers, e.g. water, ethyl alcohol, isopropyl alcohol, ethyl carbonate, ethyl acetate, benzyl alcohol, benzyl benzoate, propylene glycol, 1,3-butylene glycol, dimethylformamide, oils, especially cotton seed oil, peanut oil, corn oil, olive oil, castor oil and sesame oil, glycerol, glycerol formal, tetrahydrofurfuryl alcohol, polyethylene glycols, and fatty esters of sorbitan, or mixtures of these substances. For parenteral application, the solutions and emulsions may also be present in a sterile and blood-isotonic form.

In addition to the active substance(s), suspensions may include conventional carriers such as liquid diluents, e.g. water, ethyl alcohol, propylene glycol, suspending agents, e.g. ethoxylated isostearyl alcohols, polyoxyethylene sorbitol and sorbitan esters, microcrystalline cellulose, aluminum metahydroxide, bentonite, agar, tragacanth, or mixtures of these substances.

The drugs can be present in the form of a lyophilized sterile injectable formulation, e.g. as a sterile injectable aqueous or oily suspension. Such a suspension can also be formulated by means of methods known in the art, using suitable dispersing or wetting agents (such as Tween 80) and suspending agents. The sterile injectable formulation can also be a sterile injectable solution or suspension in a non-toxic, parenterally tolerable diluent or solvent, e.g. a solution in 1,3-butanediol. Tolerable vehicles and solvents that can be used include mannitol, water, Ringer's solution, and isotonic sodium chloride solution. Furthermore, sterile, non-volatile oils are conventionally used as solvents or suspending medium. Any mild non-volatile oil, including synthetic mono- or diglycerides, can be used for this purpose. Fatty acids such as oleic acid and glyceride derivatives thereof can be used in the production of injection agents, e.g. natural pharmaceutically tolerable oils such as olive oil or castor oil, especially in their polyoxyethylated forms. Such oil solutions or suspensions may also include a long-chain alcohol or a similar alcohol as diluent or dispersant.

The above-mentioned formulation forms may also include colorants, preservatives, as well as odor- and taste-improving additives, e.g. peppermint oil and eucalyptus oil, and sweeteners, e.g. saccharine. Preferably, the compounds according to the invention should be present in the above-mentioned pharmaceutical formulations at a concentration of about 0.1 to 99.5, more preferably about 0.5 to 95 wt.-% of the overall mixture.

In addition to the compounds of the invention, the above-mentioned pharmaceutical preparations may include further pharmaceutical active substances. The production of the pharmaceutical preparations specified above proceeds in a usual manner according to well-known methods, e.g. by mixing the active substance(s) with the carrier substance(s).

The above-mentioned preparations can be applied orally, rectally, parenterally (intravenous, intramuscular, subcutaneous routes), intracisternally, intravaginally, intraperitoneally, locally (powder, ointment, drops) in humans and animals and used in the therapy of inflammations in hollow areas and body cavities. For oral therapy, injection solutions, solutions and suspensions, gels, brew-up formulations, emulsions, ointments or drops are possible as suitable preparations. For local therapy, ophthalmic and dermatological formulations, silver and other salts, ear drops, eye ointments, powders or solutions can be used. With animals, ingestion can be effected via feed or drinking water in suitable formulations. Furthermore, gels, poudrage, powders, tablets, sustained-release tablets, premixes, concentrates, granulates, pellets, boli, capsules, aerosols, sprays and inhalants can be used in humans and animals. Furthermore, the compounds of the invention can be incorporated in other carrier materials such as plastics (plastic chains for local therapy), collagen or bone cement.

In another preferred embodiment of the invention the compounds according to the invention are incorporated in a formulation at a concentration of 0.1 to 99.5, preferably 0.5 to 95, and more preferably 20 to 80 wt.-%. That is, the compounds of the invention are present in the above-specified pharmaceutical formulations, e.g. tablets, pills, granulates and others, at a concentration of preferably 0.1 to 99.5 wt.-% of the overall mixture. The amount of active substance, i.e., the amount of a compound according to the invention that is combined with the carrier materials to produce a single dosage form, can be varied by a person skilled in the art depending on the host to be treated, the tumor to be treated, and on the particular type of administration. Once the condition of a host or patient has improved, the proportion of active compound in the preparation can be modified so as to obtain a maintenance dose. Depending on the symptoms, the dose or frequency of administration or both can subsequently be reduced to a level where the improved condition is retained. Once the symptoms have been alleviated to the desired level, the treatment should be stopped. However, patients may require an intermittent treatment on a long-term basis if any symptoms of the disease should recur. Accordingly, the proportion of the compounds, i.e. their concentration, in the overall mixture of the pharmaceutical preparation, as well as the composition or combination thereof, is variable and can be modified and adapted by a person of specialized knowledge in the art.

Those skilled in the art will be aware of the fact that the compounds of the invention can be contacted with an organism, preferably a human or an animal, on various routes. Furthermore, a person skilled in the art will also be familiar with the fact that the pharmaceutical agents in particular can be applied at varying dosages. Application should be effected in such a way that a tumor is combatted as effectively as possible, or the onset of such a disease is prevented by a prophylactic administration. Concentration and type of application can be determined by a person skilled in the art using routine tests. Preferred applications of the compounds of the invention are oral application in the form of powder, tablets, fluid mixtures, drops, capsules or the like, rectal application in the form of suppositories, solutions and the like, parenteral application in the form of injections, infusions and solutions, inhalation of vapors and aerosols, powders and pads, and local application in the form of ointments, pads, dressings, lavages and the like. Contacting with the compounds according to the invention is preferably effected in a prophylactic or therapeutic fashion. In prophylactic administration, development of the specified tumors is to be prevented at least in such a way that further propagation thereof is massively reduced, or that tumors are almost completely eliminated. In therapeutic contacting, a manifest tumor disease of a patient is already existing, and the and tumors already existing in the body should be either destroyed or inhibited in their propagation. Other forms of application preferred for this purpose are e.g. subcutaneous, sublingual, intravenous, intramuscular, intraperitoneal and/or topical ones.

In addition to the above-specified concentrations during use of the compounds of the invention, the compounds in a preferred embodiment can be employed in a total amount of 0.05 to 500 mg/kg body weight per 24 hours, preferably 5 to 100 mg/kg body weight. Advantageously, this is a therapeutic quantity which is used to prevent or improve the symptoms of a disorder or of a responsive, pathologically physiological condition. The amount administered is sufficient to inhibit tumor growth.

Obviously, the dose will depend on the age, health and weight of the recipient, degree of the disease, type of required simultaneous treatment, frequency of the treatment and type of the desired effects, and side-effects. The daily dose of 0.05 to 500 mg/kg body weight can be applied as a single dose or multiple doses in order to furnish the desired results. The dosage levels per day are applicable both in prophylaxis and treatment of a tumor disease, including infection, e.g. infections inducing or co-inducing a tumor, such as hepatitis, especially hepatitis B infection. In particular, pharmaceutical agents are typically used in about 1 to 7 administrations per day, or alternatively or additionally as a continuous infusion. Such administrations can be applied as a chronic or acute therapy. Of course, the amounts of active substance that are combined with the carrier materials to produce a single dosage form may vary depending on the host to be treated and on the particular type of administration. In a preferred fashion, the daily dose is distributed over 2 to 5 applications, with 1 to 2 tablets including an active substance content of 0.05 to 500 mg/kg body weight being administered in each application. Of course, it is also possible to select a higher content of active substance, e.g. up to a concentration of 5000 mg/kg. The tablets can also be sustained-release tablets, in which case the number of applications per day is reduced to 1 to 3. The active substance content of sustained-release tablets can be from 3 to 3000 mg. If the active substance—as set forth above—is administered by injection, the host is preferably contacted 1 to 8 times per day with the compounds of the invention or by using continuous infusion, in which case quantities of from 1 to 4000 mg per day are preferred. The preferred total amounts per day were found advantageous both in human and veterinary medicine. It may become necessary to deviate from the above-mentioned dosages, and this depends on the nature and body weight of the host to be treated, the type and severity of the disease, the type of formulation and application of the drug, and on the time period or interval during which the administration takes place. Thus, it may be preferred in some cases to contact the organism with less than the amounts mentioned above, while in other cases the amount of active substance specified above has to be surpassed. A person of specialized knowledge in the art can easily determine the optimum dosages required in each case and the type of application of the active substances.

In another particularly preferred embodiment of the invention the compounds of the invention are used in a single administration of from 1 to 80, especially from 3 to 30 mg/kg body weight. In the same way as the total amount per day, the amount of a single dose per application can be varied by a person of specialized knowledge in the art. Similarly, the compounds used according to the invention can be employed in veterinary medicine with the above-mentioned single concentrations and formulations together with the feed or feed formulations or drinking water. A single dose preferably includes that amount of active substance which is administered in one application and which normally corresponds to one whole, one half daily dose or one third or one quarter of a daily dose. Accordingly, the dosage units may preferably include 1, 2, 3 or 4 or more single doses or 0.5, 0.3 or 0.25 single doses. In a preferred fashion, the daily dose of the compounds according to the invention is distributed over 2 to 10 applications, preferably 2 to 7, and more preferably 3 to 5 applications. Of course, continuous infusion of the agents according to the invention is also possible.

In a particularly preferred embodiment of the invention, 1 to 10 tablets or capsules, preferably 4 to 8 capsules or tablets, and more preferably 6 capsules or tablets are administered in each oral application of the compounds of the invention. The tablets according to the invention can be provided with coatings and envelopes well-known to those skilled in the art or can be composed in a way so as to release the active substance(s) only in preferred, particular regions of the host.

In another preferred embodiment of the invention the compounds according to the invention can be employed together with at least one other well-known pharmaceutical agent. That is to say, the compounds of the invention can be used in a prophylactic or therapeutic combination in connection with well-known drugs. Such combinations can be administered together, e.g. in an integrated pharmaceutical formulation, or separately, e.g. in the form of a combination of tablets, injection or other medications administered simultaneously or at different times, with the aim of achieving the desired prophylactic or therapeutic effect. These well-known agents can be agents which enhance the effect of the compounds according to the invention.

Of course, it is also possible to use the compounds of the invention, particularly the pharmaceutical agents, alone or together with other agents in a therapy, e.g. in a combination therapy, as a regional therapy; this can be preferred in the event of a liver tumor, for example.

It is well-known to those skilled in the art that lowering the concentration of glutathione by oxidizing agents or adduct-forming agents can be advantageous in particular tumor diseases. To this end, it can be preferred to increase the concentration of platinum complexes as chemotherapeutic agent or of the compounds according to the invention.

Typically, there is an optimum ratio of compound(s) of the invention with respect to each other and/or with respect to other therapeutic or effect-enhancing agents (such as transport inhibitors, metabolic inhibitors, inhibitors of renal excretion or glucuronidation, such as probenecid, acetaminophen, aspirin, lorazepan, cimetidine, ranitidine, colifibrate, indomethacin, ketoprofen, naproxen etc.) where the active substances are present at an optimum ratio. Optimum ratio is defined as the ratio of compound(s) of the invention to other therapeutic agent(s) where the overall therapeutic effect is greater than the sum of the effects of the individual therapeutic agents. In general, the optimum ratio is found when the agents are present at a ratio of from 10:1 to 1:10, from 20:1 to 1:20, from 100:1 to 1:100 and from 500:1 to 1:500. In some cases, an exceedingly small amount of a therapeutic agent will be sufficient to increase the effect of one or more other agents. In addition, the use of the compounds of the invention in combinations is particularly beneficial in order to reduce the risk of developing resistance and/or increase the therapeutic effectiveness. Of course, the compounds of the invention can be used in combination with other well-known anti-tumor agents. Such agents are well-known to those skilled in the art. Accordingly, the compounds of the invention can be administered together with all conventional agents, especially other drugs, available for use particularly in connection with tumor drugs, either as a single drug or in a combination of drugs. They can be administered alone or in combination with same.

In a preferred fashion the compounds of the invention are administered together with said other well-known pharmaceutical agents at a ratio of about 0.005 to 1. Preferably, the compounds of the invention are administered particularly together with virus-inhibiting agents at a ratio of from 0.05 to about 0.5 parts and up to about 1 part of said known agents. The pharmaceutical composition can be present in substance or as an aqueous solution together with other materials such as preservatives, buffer substances, agents to adjust the osmolarity of the solution, and so forth.

In a preferred fashion the pharmaceutical agent is employed as a vaccine after tumor formation, or as a preventive vaccination. Advantageously, vaccination is effected in such a way that, following application, a protection against spreading or formation of tumors is developed in the organism. Of course, it is also possible to effect vaccination immediately prior to or shortly after manifestation of a tumor, or as a therapy with a plurality of applications. Those skilled in the art are familiar with the fact that tumor treatment can be advantageous at virtually any point in time following formation of metastases, so that vaccination in the meaning of the invention could also be application of the inventive pharmaceutical agent weeks, months, years or decades after formation of a tumor.

The invention also relates to a kit and to the use thereof in medicine. In a preferred fashion, the compounds of the invention or the kit comprising same are used in a combination therapy, especially in the treatment of tumors. In a particularly preferred fashion, said combination therapy comprises a chemotherapy, a treatment with cytostatic agents and/or a radiotherapy. In a particularly preferred embodiment of the invention the combination therapy is an adjuvant, biologically specific form of therapy, and in a particularly preferred fashion, said form of therapy is an immune therapy. Furthermore, in a particularly preferred fashion the combination therapy comprises a gene therapy and/or a therapy using a compound according to the invention. Various combination therapies, especially for the treatment of tumors, are well-known to those skilled in the art. For example, a treatment with cytostatic agents or e.g. irradiation of a particular tumor area can be envisaged within the scope of a combination therapy, and this treatment is combined with a gene therapy, using the compounds of the invention as anticancer agents. Accordingly, the use of the compounds according to the invention for increasing the sensitivity of tumor cells to cytostatic agents and/or radiation can be particularly preferred. Furthermore, a preferred use of the compounds according to the invention is in inhibiting the vitality, the proliferation rate of cells and/or inducing apoptosis and cell cycle arrest.

In a preferred embodiment the cancerous disease or tumor being treated or prevented is selected from the group of cancerous diseases or tumor diseases of the ear-nose-throat region, of the lungs, mediastinum, gastrointestinal tract, urogenital system, gynecological system, breast, endocrine system, skin, bone and soft-tissue sarcomas, mesotheliomas, melanomas, neoplasms of the central nervous system, cancerous diseases or tumor diseases during infancy, lymphomas, leukemias, paraneoplastic syndromes, metastases with unknown primary tumor (CUP syndrome), peritoneal carcinomatoses, immunosuppression-related malignancies and/or tumor metastases.

More specifically, the tumors may comprise the following types of cancer: adenocarcinoma of breast, prostate and colon; all forms of lung cancer starting in the bronchial tube; bone marrow cancer, melanoma, hepatoma, neuroblastoma; papilloma; apudoma, choristoma, branchioma; malignant carcinoid syndrome; carcinoid heart disease, carcinoma (for example, Walker carcinoma, basal cell carcinoma, squamobasal carcinoma, Brown-Pearce carcinoma, ductal carcinoma, Ehrlich tumor, in situ carcinoma, cancer-2 carcinoma, Merkel cell carcinoma, mucous cancer, non-parvicellular bronchial carcinoma, oat-cell carcinoma, papillary carcinoma, scirrhus carcinoma, bronchio-alveolar carcinoma, bronchial carcinoma, squamous cell carcinoma and transitional cell carcinoma); histiocytic functional disorder; leukemia (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia); malignant histiocytosis, Hodgkin disease, non-Hodgkin lymphoma, solitary plasma cell tumor; reticuloendotheliosis, chondroblastoma; chondroma, chondrosarcoma; fibroma; fibrosarcoma; giant cell tumors; histiocytoma; lipoma; liposarcoma; leukosarcoma; mesothelioma; myxoma; myxosarcoma; osteoma; osteosarcoma; Ewing sarcoma; synovioma; adenofibroma; adenolymphoma; carcinosarcoma, chordoma, craniopharyngioma, dysgerminoma, hamartoma; mesenchymoma; mesonephroma, myosarcoma, ameloblastoma, cementoma; odontoma; teratoma; thymoma, chorioblastoma; adenocarcinoma, adenoma; cholangioma; cholesteatoma; cylindroma; cystadenocarcinoma, cystadenoma; granulosa cell tumor; gynadroblastoma; hidradenoma; islet-cell tumor; Leydig cell tumor; papilloma; Sertoli cell tumor, theca cell tumor, leiomyoma; leiomyosarcoma; myoblastoma; myoma; myosarcoma; rhabdomyoma; rhabdomyosarcoma; ependymoma; ganglioneuroma, glioma; medulloblastoma, meningioma; neurilemmoma; neuroblastoma; neuroepithelioma, neurofibroma, neuroma, paraganglioma, non-chromaffin paraganglioma, angiokeratoma, angiolymphoid hyperplasia with eosinophilia; sclerotizing angioma; angiomatosis; glomangioma; hemangioendothelioma; hemangioma; hemangiopericytoma, hemangiosarcoma; lymphangioma, lymphangiomyoma, lymphangiosarcoma; pinealoma; cystosarcoma phylloides; hemangiosarcoma; lymphangiosarcoma; myxosarcoma, ovarian carcinoma; sarcoma (for example, Ewing sarcoma, experimentally, Kaposi sarcoma and mast cell sarcoma); neoplasms (for example, bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of the head and neck, of the central nervous system, neoplasms of the hearing organ, pelvis, respiratory tract and urogenital tract); neurofibromatosis and cervical squamous cell dysplasia.

In another preferred embodiment the cancerous disease or tumor being treated or prevented is selected from the group of tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarian carcinoma, tumors of the uterine tube (Tuba Faloppii), tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, morbus Hodgkin, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancy such as Kaposi sarcoma, AIDS-associated lymphomas, AIDS-associated lymphomas of the central nervous system, AIDS-associated morbus Hodgkin and AIDS-associated anogenital tumors, transplantation-related malignancy, metastasized tumors comprising brain metastases, lung metastases, liver metastases, bone metastases, pleural and pericardial metastases, and malignant ascites.

In another preferred embodiment the cancerous disease or tumor being treated or prevented is selected from the group comprising mammary carcinomas, gastrointestinal tumors, including colon carcinomas, stomach carcinomas, pancreas carcinomas, colon cancer, small intestine cancer, ovarian carcinomas, cervical carcinomas, lung cancer, prostate cancer, kidney cell carcinomas and/or liver metastases.

Without intending to be limiting, the invention will be explained in more detail with reference to the following examples.

1. Preparation of Chelidonine Acetate

1 g of chelidonine is dissolved in 10 ml of dry pyridine and added with 2 ml of acetic anhydride. Following standing at room temperature (24 h), the mixture is poured into 100 ml of ice water, the precipitated raw product is extracted with ether, and the ether phase is repeatedly washed with water. Subsequently, the solvent is removed in vacuum, and the remaining raw product is recrystallized from ethanol.

Yield: 0.9 g (about 85% of theoretical amount).

2. Preparation of the Chelidonine Esters

General Protocol:

300 mg of chelidonine is dissolved in 30 ml of dry chloroform and added with a 1.2-fold molar amount of the respective acid chloride.

Following addition of 3 ml of pyridine, the reaction mixture is allowed to stand at room temperature overnight.

Work-Up/Purification:

Following addition of another 100 ml of chloroform, the organic phase is washed with water 4 to 5 times. The solvent is removed in a rotary evaporator, and the remaining raw product is recrystallized from ethanol once or twice (recrystallization: ˜−20° C.).

2.1. Preparation of Chelidoninyl Trifluoroacetate (A101)

Batch: 300 mg of chelidonine, 380 mg of trifluoroacetic anhydride

Yield: 300 mg (81% of theoretical amount); m.p.=140-42° C.

2.2. Preparation of Chelidoninyl Trichloromethyl Carbonate (A102)

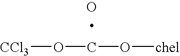

Batch: 300 mg of chelidonine, 270 mg of trichloromethyl chloroformate

Yield: 200 mg (48.1% of theoretical amount); m.p.=154-57° C.

2.3. Preparation of Succinic Acid Chelidoninyl Methyl ester (A103)

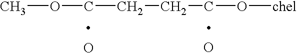

Batch: 300 mg of chelidonine, 300 mg of succinic acid methyl ester chloride

Yield: 200 mg (52.9 of theoretical amount); m.p.=84-85° C.

3. Preparation of Chelidoninyl Ethyl Oxalate

3.1. General Protocols for the Reaction with Carbonic Acid Chlorides and Anhydrides
Chelidonine monohydrate and the double molar amount of the corresponding carboxylic acid chloride or anhydride (10% excess) are weighed in an Erlenmeyer flask.
Add about 10 ml of pyridine.
Shake reaction batch and allow to stand for 3 days at room temperature.
Place mixture in separating funnel, add about 20 ml of ether, then wash with water 5 to 6 times.
Evaporate solvent.
Purification.

Reaction:

Reaction of Chelidonine Monohydrate (Fluka Ch:425201/1) with Oxalic Ester Chloride (Lancaster):

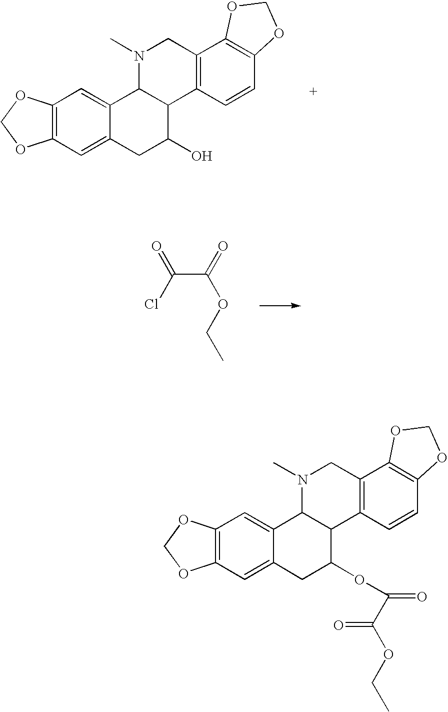

Chelidonine monohydrate: m.w.=371.39 g/mol

Oxalic ester chloride: m.w.=136.53 g/mol

Chelidoninyl ethyl oxalate: m.w.=453.42 g/mol, C₂₄H₂₃NO₈

Yield: 25.9% (80.4 mg)
Initial weight: 0.3106 g of chelidonine monohydrate and 0.2805 g of oxalic ester chloride.
Flash chromatography (1% triethylamine in heptane-ether mixture 8:2 v/v).

The reaction and the preparation of chelidoninyl ethyl oxalate (S2) are shown in the IR spectrum and mass spectrum (FIGS. 1 and 2).

4. Preparation of N-(3-trifluoromethylphenyl)-chelidoninylurethane

4.1. General Protocols for the Reaction with Isocyanates and Isothiocyanates
Chelidonine monohydrate and the double molar amount of the corresponding isocyanate or isothiocyanate (10% excess) are weighed in a single-necked flask.
Add about 40 ml of acetonitrile.
Boil reaction batch for 4 h at reflux.
Evaporate solvent.
Purification.

Reaction:

Reaction of chelidonine monohydrate (Fluka Ch:425201/1) with 3-trifluoromethylphenylisocyanate (Riedel-de Haën AG)

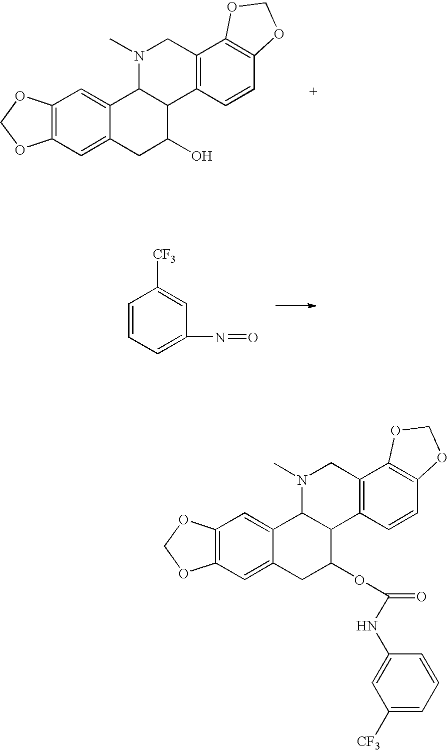

Chelidonine monohydrate: m.w.=371.39 g/mol

Trifluoromethylphenylisocyanate: m.w.=187.11 g/mol

N-(3-Trifluoromethylphenyl)chelidoninylurethane: m.w.=540.47 g/mol, C₂₈H₂₃F₃N₂O₆

Yield: 34.8% (104.4 mg)
Initial weight: 0.3001 g of chelidonine monohydrate and 0.3502 g of N-(3-trifluoromethylphenyl)chelidoninylurethane.
Column chromatography (5% CH₂Cl₂ in a heptane-ethyl acetate mixture 8:3 v/v).
Recrystallization in toluene.

The preparation of N-(3-trifluoromethylphenyl)chelidoninylurethane (S5) is shown with reference to the IR and mass spectra (FIGS. 3 and 4).

5. Preparation of Phenylalanine Chelidoninyl Ester

5.1. General Protocols for the Reaction with FMOC-Protected Amino Acids
Chelidonine monohydrate, an equimolar amount of the FMOC-protected amino acid and the double molar amount of dicyclohexylcarbodiimide (10% excess) are weighed in a single-necked flask.
Add about 50 ml of ethyl acetate.
Stir reaction batch for 3 days at room temperature.
Evaporate ethyl acetate.
Removal of dicyclohexylurea: add carbon tetrachloride, heat, and filtrate while hot (the residue is dicyclohexylurea).
Evaporate carbon tetrachloride.
Removal of the protective group: add about 4 ml of morpholine and allow to stand for 30 minutes.
Evaporate morpholine.
Purification.

Reaction:

Reaction of chelidonine monohydrate (Fluka Ch:425201/1) with N-(9-fluorenylmethyloxycarbonyl)-L-phenylalanine (Aldrich)

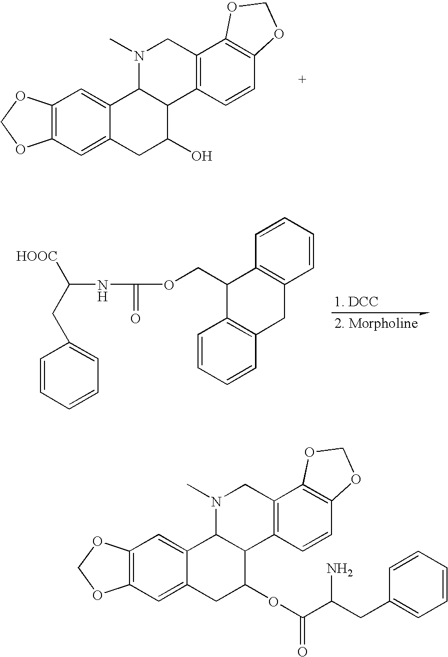

Chelidonine monohydrate: m.w.=371.39 g/mol

N-(9-Fluorenylmethyloxycarbonyl)-L-phenylalanine: m.w.=387.44 g/mol

Dicyclohexylcarbodiimide (DCC): m.w.=206.33 g/mol

Phenylalanine chelidoninyl ester: m.w.=500.52 g/mol, C₂₉H₂₈N₂O₆

Yield: 41.1% (113.9 mg)
Initial weight: 0.277 g of chelidonine monohydrate, 0.2902 g of N-(9-fluorenylmethyloxycarbonyl)-L-phenylalanine and 0.3662 g of dicyclohexylcarbodiimide
Flash chromatography (1% triethylamine in a heptaneethyl acetate mixture 7:3 v/v)

The preparation of phenylalanine chelidoninyl ester (S9) is shown with reference to the IR and mass spectra (FIGS. 5 and 6).

6. Preparation of Proline Chelidoninyl Ester

6.1. See General Protocols for the Reaction with FMOC-Protected Amino Acids

Reaction:

Reaction of chelidonine monohydrate (Fluka Ch:425201/1) with N-(9-fluorenylmethyloxycarbonyl)-L-proline (Aldrich)

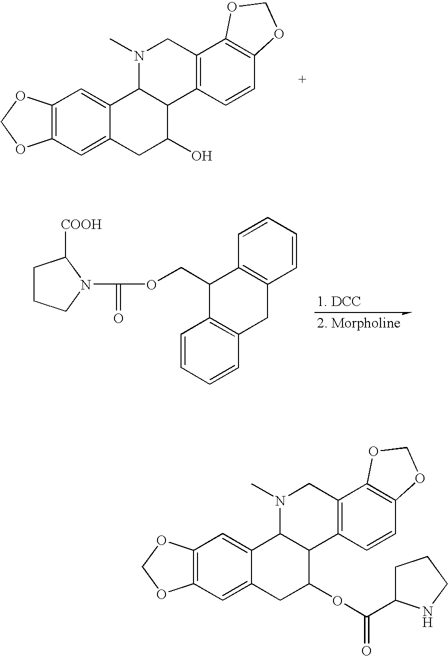

Chelidonine monohydrate: m.w.=371.39 g/mol

N-(9-Fluorenylmethyloxycarbonyl)-L-proline: m.w.=337.38 g/mol

Dicyclohexylcarbodiimide (DCC): m.w.=206.33 g/mol

Proline chelidoninyl ester: m.w.=450.47 g/mol, C₂₅H₂₆N₂O₆

Yield: 36.3 (101.3 mg)
Initial weight: 0.2789 g of chelidonine monohydrate, 0.2790 g of N-(9-fluorenylmethyloxycarbonyl)-L-proline and 0.3705 g of dicyclohexylcarbodiimide
Flash chromatography (1% triethylamine in a heptaneethyl acetate mixture 7:3 v/v)

The preparation of proline chelidoninyl ester (S10) is shown with reference to the IR and mass spectra (FIGS. 7 and 8).

7. Preparation of Alanine Chelidoninyl Ester

7.1. See General Protocols for the Reaction with FMOC-Protected Amino Acids

Reaction:

Reaction of chelidonine monohydrate (Fluka Ch:425201/1) with N-(9-fluorenylmethyloxycarbonyl)-L-alanine (Aldrich)

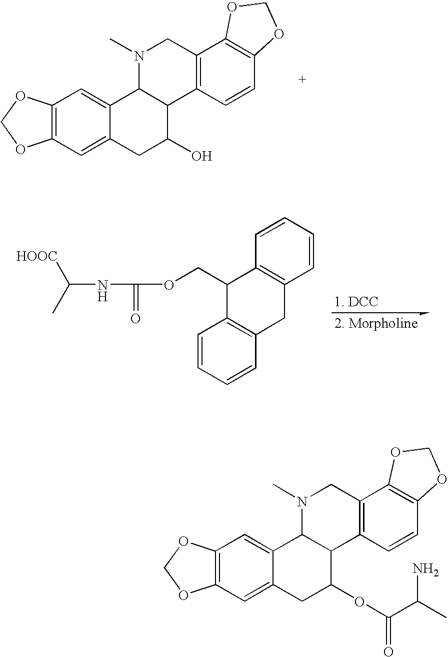

Chelidonine monohydrate: m.w.=371.39 g/mol

N-(9-Fluorenylmethyloxycarbonyl)-L-alanine: m.w.=311.32 g/mol

Dicyclohexylcarbodiimide (DCC): m.w.=206.33 g/mol

Alanine chelidoninyl ester: m.w.=424.43 g/mol, C₂₃H₂₄N₂O₆

Yield: 33.5% (101.0 mg)
Initial weight: 0.3015 g of chelidonine monohydrate, 0.2660 g of N-(9-fluorenylmethyloxycarbonyl)-L-alanine and 0.3698 g of dicyclohexylcarbodiimide
Flash chromatography (1% triethylamine in a heptaneethyl acetate mixture 7:3 v/v)

The preparation of alanine chelidoninyl ester (S11) is shown with reference to the IR and mass spectra (FIGS. 9 and 10).

The following results were obtained in an MTT cytotoxicity test. The values are IC₅₀ values in ng/ml. As can be seen, chelidoninyl trichloromethyl carbonate has similar activity as native chelidonine; chelidonine trifluoroacetate and chelidoninyl methyl succinate have activities that are 5 times higher (ranging from 4-10 times) and approximately 30 times higher (ranging from 50-150 times), respectively, compared to native chelidonine.

| | | | Chelidoninyl | Chelidoninyl |
|---|---|---|---|---|
| | | Chelidonine | trichloromethyl | methyl |
| | Chelidonine | trifluoroacetate | carbonate | succinate |
| T47D | 200 | 20 | 200 | 20 |
| (breast) | | | | |
| Colo205 | 370 | 45 | 500 | 3.5 |
| (colon) | | | | |
| Panc1 | 250 | 60 | 250 | 5 |
| (pancreas) | | | | |
| BxPC3 | >8000 | 30 | >8000 | <3.5 |
| (pancreas) | | | | |
| U373MG | >8000 | >4000 | >8000 | 4000 |
| (astrocyte) | | | | |
| SK-0V3 | not | 60 | not | 3.5 |
| (ovarian) | tested | | tested | |

1. New chelidonine derivatives having an anti-tumoral effect, selected from the group consisting of chelidoninyl trifluoroacetate, chelidoninyl trichloromethyl carbonate, chelidoninyl methyl succinate, chelidoninyl ethyl oxalate, N-(3-trifluoromethylphenyl)chelidoninylurethane, phenylalanine chelidoninyl ester, proline chelidoninyl ester and alanine chelidoninyl ester.

2. The chelidonine derivatives according to claim 1, wherein
the anti-tumoral effect is modulation of cell growth, cell differentiation and/or cell division.

3. A pharmaceutical agent comprising at least one chelidonine derivative according to claim 1, optionally together with a tolerable pharmaceutical carrier, adjuvant and/or vehicle.

4. The pharmaceutical agent according to claim 3, wherein
the carriers are fillers, diluents, binders, humectants, disintegrants, dissolution retarders, absorption enhancers, wetting agents, adsorbents and/or lubricants.

5. The pharmaceutical agent according to claim 3, wherein
the carriers are liposomes, siosomes and/or niosomes.

6. Method for prophylaxis, therapy, follow-up and aftercare of diseases associated with cell growth, cell differentiation and/or cell division comprising administering to an organism in need thereof a chelidonine derivative chosen from the group consisting of chelidonine acetate, chelidoninyl trifluoroacetate, chelidoninyl trichloromethyl carbonate, chelidoninyl methyl succinate, chelidoninyl ethyl oxalate, N-(3-trifluoromethylphenyl)chelidoninylurethane, phenylalanine chelidoninyl ester, proline chelidoninyl ester, alanine chelidoninyl ester and combinations thereof in a prophylaxis, therapy, follow-up and aftercare of diseases associated with cell growth, cell differentiation and/or cell division effective amount.

7. The method of claim 6, wherein one of said diseases is a tumor disease.

8. The method of claim 6, wherein
the tumor diseases are selected from the group of neoplastic tumors, inflammatory tumors, abscesses and combinations thereof, effusions and edema.

9. The method of claim 7, wherein the tumor disease comprises a solid tumor or leukemia.

10. The method of claim 9, wherein
the solid tumor is a tumor of the urogenital tract and/or gastrointestinal tract.

11. The method of claim 7, wherein the tumor disease is a colon carcinoma, stomach carcinoma, pancreas carcinoma, small intestine carcinoma, ovarian carcinoma, cervical carcinoma, lung carcinoma, prostate carcinoma, mammary carcinoma, renal cell carcinoma, a brain tumor, head-throat tumor, liver carcinoma, and/or a metastase of the above tumors.

12. The method of claim 9, wherein
the solid tumor is a mammary, bronchial, colorectal, and/or prostate carcinoma and/or a metastase of the above tumors.

13. The method of claim 10, wherein
the tumor of the urogenital tract is a bladder carcinoma and/or a metastase of such tumors.

14. The method of claim 6, wherein
said follow-up is monitoring the effectiveness of an anti-tumor treatment.

15. Method for prophylaxis, prevention, diagnosis, attenuation, therapy, follow-up and/or aftercare of metastasizing, invasion and/or angiogenesis comprising administering to an organism in need thereof
at least one chelidonine derivative according to claim 1 in a prophylaxis, prevention, diagnosis, attenuation, therapy, follow-up and/or aftercare of metastasizing, invasion and/or angiogenesis effective amount.

16. The method of claim 15, wherein
said follow-up is monitoring the effectiveness of an anti-tumor treatment.

17. The method of claim 15, wherein
said method is part of a combination therapy.

18. The method of claim 17, wherein
said combination therapy comprises a chemotherapy, a treatment with cytostatic agents and/or a radiotherapy.

19. The method of claim 17, wherein
the combination therapy comprises an adjuvant, biologically specified form of therapy.

20. The method of claim 19, wherein
said form of therapy is an immune therapy.

21. The method of claim 6, wherein the method increases sensitivity of tumor cells to cytostatic agents and/or radiation.

22. The method of claim 6, wherein the method inhibits viability, proliferation rate of cells in order to induce apoptosis and/or cell cycle arrest.

23. The method of claim 15, wherein said
at least one chelidonine derivative according to claim 1 is prepared as gel, poudrage, powder, tablet, sustained-release tablet, premix, emulsion, brew-up formulation, drops, concentrate, granulate, syrup, pellet, bolus, capsule, aerosol, spray and/or inhalant and/or inhalant and applied in this form.

24. The method of claim 15, wherein
at least one chelidonine derivative according to claim 1 is present in a preparation at a concentration of from 0.1 to 99.5, preferably from 0.5 to 95.0, and more preferably from 20.0 to 80.0 weight percent.

25. The method of claim 6, wherein the chelidonine derivative according to claim 1 is administered orally, subcutaneously, intravenously, intramuscularly, intraperitoneally, topically via injection, vaginally, rectally and/or nasally.

26. The method of claim 6, wherein the
at least one chelidonine derivative according to claim 1 is administered in overall amounts of from 0.05 to 500 mg per kg, preferably from 5 to 100 mg per kg body weight per 24 hours.

27. A method for the preparation of the chelidonine derivatives according to claim 1
comprising reacting chelidonine with pyridine and acetic anhydride to obtain chelidonine acetate.

28. A method for the preparation of the chelidonine derivatives according to claim 1, wherein
a mixture of chelidonine, pyridine and acetic anhydride is incubated for at least 12 hours at room temperature and this mixture is subsequently poured in ice water, so that a raw product precipitates, and the raw product is extracted with ether.

29. A method for the preparation of the chelidonine derivatives according to claim 1, wherein
chelidoninyl trifluoroacetate, chelidoninyl trichloromethyl carbonate, and/or chelidoninyl methyl succinate are obtained by reacting chelidonine with chloroform and the respective acid chloride, the mixture of chelidonine, chloroform and the respective acid chloride being added with pyridine, and the resulting mixture being incubated for at least 4 hours at room temperature.

30. A method for the preparation of the chelidonine derivatives according to claim 1, wherein chelidonine monophosphate is reacted with oxalic ester chloride to obtain chelidoinyl ethyl oxalate.

31. A method for the preparation of the chelidonine derivatives according to claim 1, wherein
chelidonine monohydrate is reacted with 3-trifluoromethylphenylisocyanate to obtain N-(3-trifluoromethylphenyl)chelidoninylurethane.

32. A method for the preparation of the chelidonine derivatives according to claim 1, wherein chelidonine monohydrate is reacted with N-(9-fluorenylmethyloxycarbonyl)-L-phenylalanine to obtain phenylalanine chelidoninyl ester.

33. A method for the preparation of the chelidonine derivatives according to claim 1, wherein chelidonine monohydrate is reacted with N-(9-fluorenylmethyloxycarbonyl)-L-proline to obtain proline chelidoninyl.

34. A method for the preparation of the chelidonine derivatives according to claim 1, wherein chelidonine monohydrate is reacted with N-(9-fluorenylmethyloxycarbonyl)-L-alanine to obtain alanine chelidoninyl ester.

35. The method of claim 6, wherein said organism is a human or an animal.

36. (canceled)

37. A method for the production of a pharmaceutical agent for the treatment of a tumor disease,
wherein
at least one chelidonine derivative according to claim 1 is employed together with a pharmaceutically tolerable carrier.

38. A kit comprising at least one chelidonine derivative according to claim 1, optionally together with information for combining the contents of the kit.

39. The kit according to claim 38, wherein said kit is used for prophylaxis or therapy of tumor diseases.
